(19) United States
(12) Patent Application Publication
(10) Pub. No.: US 2007/0240491 A1
(43) Pub. Date: Oct. 18, 2007

(54) Hydrogen Sensor

(75) Inventors: Igor Pavlovsky, Cedar Park, TX (US); Richard Lee Fink, Austin, TX (US); Zvi Yaniv, Austin, TX (US)

Correspondence Address: FISH & RICHARDSON P.C., P.O BOX 1022, Minneapolis, MN, 55440-1022, US

(73) Assignee: NANO-PROPRIETARY, INC., Austin, TX (US)

(21) Appl. No.: 11/737,586

(22) Filed: Apr. 19, 2007

Related U.S. Application Data
(63) Continuation-in-part of application No. 11/551,630, filed on Oct. 20, 2006.
(63) Continuation-in-part of application No. 10/854,420, filed on May 26, 2004.
(60) Provisional application No. 60/728,980, filed on Oct. 21, 2005.
(60) Provisional application No. 60/475,558, filed on Jun. 3, 2003.
(60) Provisional application No. 60/793,377, filed on Apr. 20, 2006.

(51) Int. Cl. G01N 33/00; G01N 27/00

(52) U.S. Cl. 73/31.05

(57) ABSTRACT

A nanoparticle based sensor in which smaller particles are seeded at a higher density to produce a faster response time than that of a sensor using larger particles and less dense seeding. The nanoparticles may comprise palladium nanoparticles. The sensor may be used in hydrogen fuel cells.

This application is a continuation-in-part of U.S. patent application Ser. No. 11/551,630, which claims priority to U.S. Provisional Application Ser. No. 60/728,980 and PCT Application PCT/US2006/030314, all of which are hereby incorporated by reference herein. This application is a continuation-in-part of U.S. patent application Ser. No. 10/854,420, which claims priority to the U.S. Provisional Application Ser. No. 60/475,558 and PCT Application PCT/US2006/030314, which claims priority to the following provisional patent applications: Ser. Nos. 60/728,353 and 60/728,980, all of which are hereby incorporated by reference herein. This application also claims priority to U.S. Provisional Application Ser. No. 60/793,377, which is hereby incorporated by reference herein.

### BACKGROUND

Sensors using palladium metal for gaseous hydrogen sensing is a two step process, wherein the diatomic hydrogen molecule dissociates into monoatomic hydrogen in the surface of the palladium metal and the monoatomic hydrogen diffuses into the palladium lattice causing a lattice expansion in palladium (up to 5%), triggering a phase change (see FIG. 1). When a thin film of palladium is placed between two electrical contacts, the resistance of the film increases on exposure to hydrogen due to the phase change. Their turn on time (response time) is generally not fast enough for commercial applications, for example for use in a hydrogen fuel cell.

### BRIEF DESCRIPTION OF THE DRAWINGS

FIG. 1 illustrates a graph showing a thin film hydrogen sensor with a phase transition in palladium;

FIG. 2 illustrates a variation in current within a hydrogen sensor;

FIG. 3 illustrates a schematic diagram of a hydrogen sensor on a resistive substrate, with the arrows showing the direction of the current flow, wherein the resistors represent the substrate;

FIG. 4 illustrates a two-step palladium nanoparticle plating process on a resistive substrate;

FIG. 5 illustrates a table showing particle size and density variations in nanoparticles in accordance with embodiments of the present invention;

FIG. 6 illustrates representative SEM micrographs showing particle size and density variations of embodiments of the present invention;

FIG. 7 illustrates a graph of the response of sensors to 40,000 ppm hydrogen at 60° C. in accordance with embodiments of the present invention;

FIG. 8 illustrates a graph of a response of sensors to 400 ppm of hydrogen at 60° C.;

FIG. 9 illustrates a top view schematic showing a diameter (d) to interparticle distance (l) between two adjacent palladium nanoparticles in accordance with embodiments of the present invention;

FIG. 10A illustrates a sensor element in accordance with embodiments of the present invention;

FIG. 10B illustrates a sensor pair with a titanium reference element in accordance with embodiments of the present invention;

FIG. 10C illustrates a sensor pair, wire-bonded to a carrier PC board in accordance with embodiments of the present invention;

FIG. 10D illustrates a solid-pattern active element in accordance with embodiments of the present invention;

FIG. 10E illustrates a striped-pattern active element in accordance with embodiments of the present invention;

FIG. 11 illustrates operation of a sensor;

FIG. 12 illustrates an apparatus for testing the sensors;

FIGS. 13(a)-(b) illustrate a change of resistance of hydrogen sensors;

FIGS. 14(a)-(b) illustrate initial resistances of sensors; and

FIG. 15 illustrates sensor response for temperature and concentration.

### DETAILED DESCRIPTION

A problem to be solved is to find a range of particle size and density for a fast hydrogen gas sensor. Disclosed herein is a range of particle size and density that achieves a response time of 10 seconds or lesser at high hydrogen concentrations.

Another approach has been disclosed in U.S. Pat. No. 6,849,911, which is incorporated by reference herein, for the creation of a palladium based hydrogen sensor, by fabricating a network of palladium nanoparticles on a resistive substrate by an electrochemical deposition technique. As the palladium nanoparticles expand on a resistive substrate between two electrical contacts, they short out miniscule resistances in the resistive substrate which happen to lie beneath two adjacent nanoparticles. On a large-scale statistical basis, the end-to-end resistance of the substrate then decreases in proportion to the amount of hydrogen. This sensor therefore measures hydrogen, rather than just detecting its presence.

(a) Palladium Nanoparticles Networks Versus Thin Films or Nanowires (Prior Art)

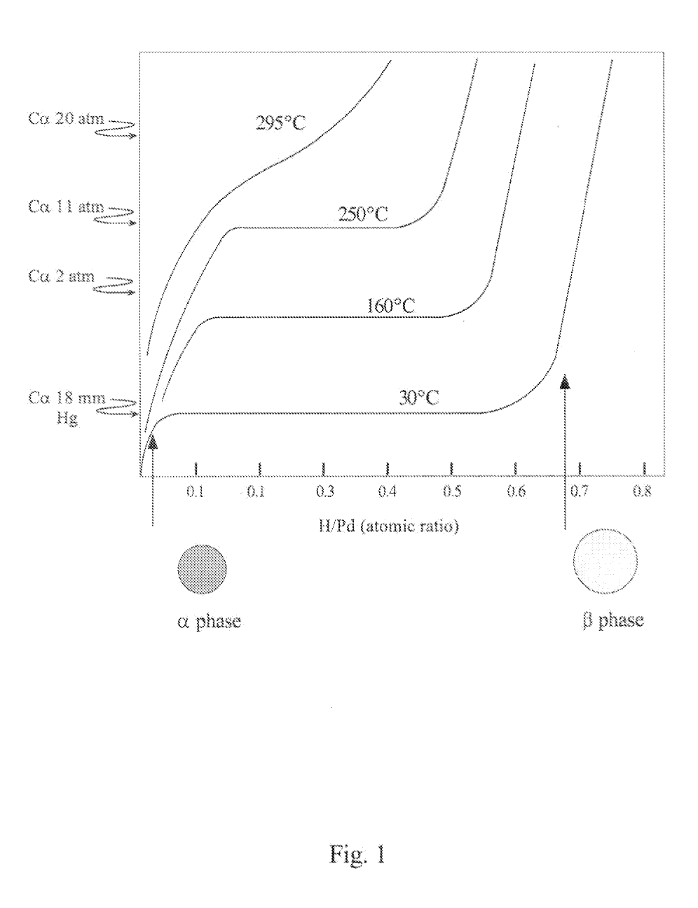

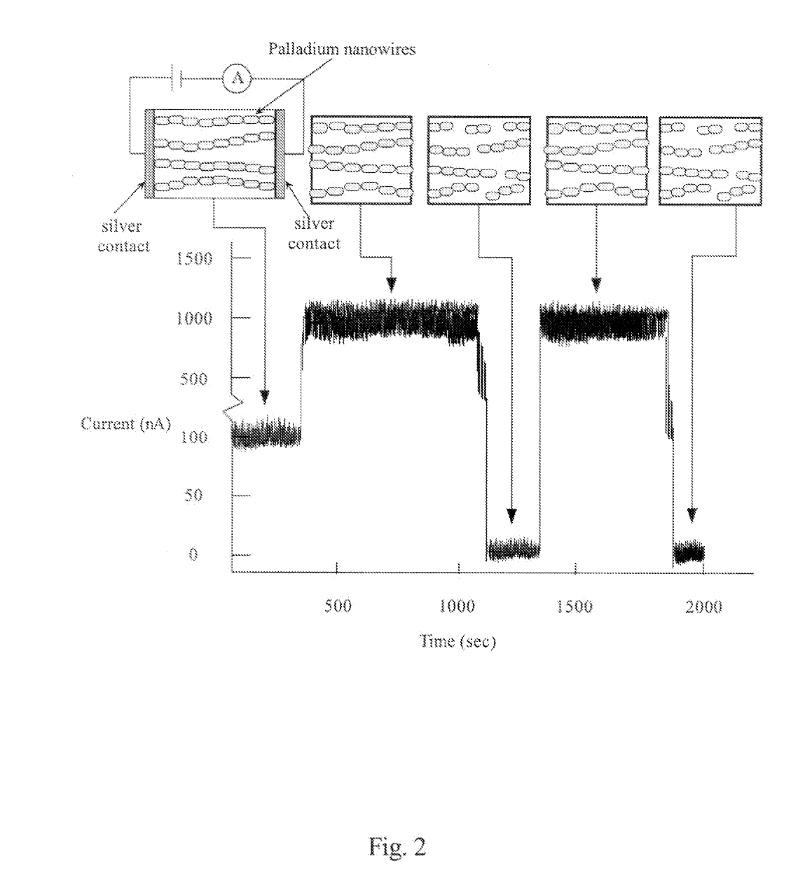

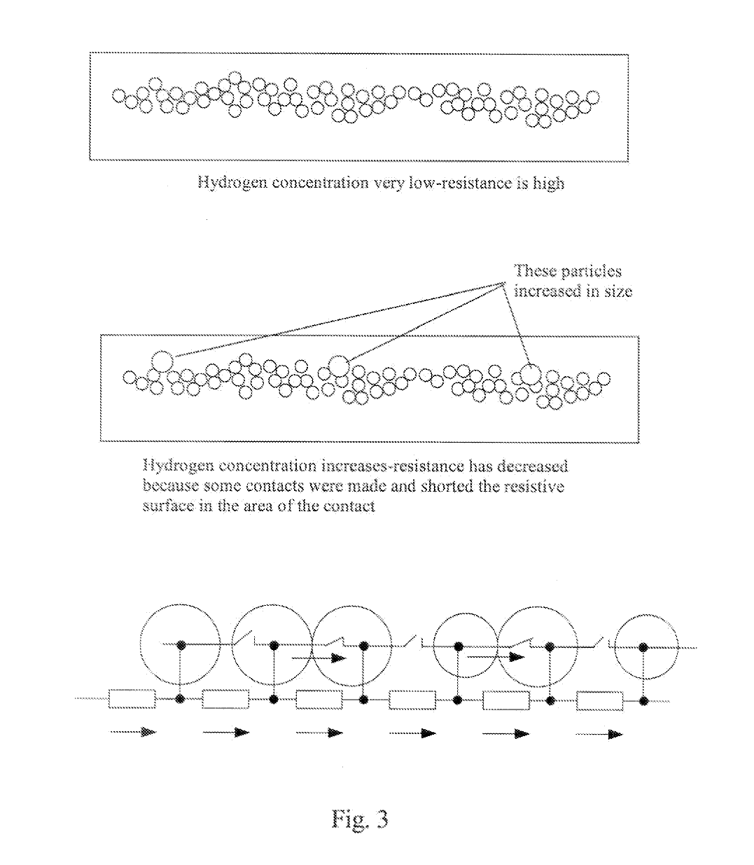

A thin film of palladium is a continuous surface, with normal metallic connection between atoms. The response of thin-film palladium to increasing levels of hydrogen has a positive coefficient. That is, the resistance increases with increasing hydrogen concentrations (see FIG. 1). The resistance of a palladium nanowire decreases (see FIG. 2) with increasing exposure to hydrogen, and similar to a low-resistance switch. The switch is closed when the nanoparticles expand and touch each other along the entire length of the wire. It is relatively insensitive to gradations in concentration. The resistive response of the palladium nanoparticle networks is a gradual decrease in resistance upon increasing exposure to hydrogen (see FIG. 3).

(b) Use of a Resistive Substrate and Palladium ‘Nano-Switches’ (Prior Art: U.S. Pat. No. 6,849,911)

Uses nanoparticles on a resistive substrate as known in prior art (see FIG. 3), such that the nanoparticles do not touch each other for the most part before exposure to hydrogen. Upon exposure to hydrogen, the particles expand in size and begin to touch each other causing electrical shorts on the resistive substrate to which they are attached, incrementally reducing the overall end-to-end resistance of the substrate. Because the particles form a random network and are of random size, the shorting does not occur at a specific concentration of hydrogen, as for the case of nanowires. Rather, the overall resistance gradually decreases as the exposed hydrogen concentration increases.

(c) Characteristics of a Proper Resistive Layer (Prior Art: U.S. Pat. No. 6,849,911)

Certain requirements are imposed on the resistive layer on which the nanoparticles are formed. It should ideally be stable with temperature, should be insensitive to environmental factors, should accept the formation of the nanoparticles. It further yields a certain ‘non-exposed’ resistance that is optimal for the electronics to which it connects. For the case of the sensors and electronics, the optimum resistance of a 0.5 mm×2.0 mm resistive surface yields a resistance range of 1200 to 2200 Ohms.

The optimum value is determined by desired operating current, impedance-based immunity to nearby electrical signals, and by resistive stability of the surface. If a surface such as titanium is used, thicker surface films improve aging characteristics but diminish both resistance and available signal. If that same film is too thin, electrical noise increases and the film is less immune to effects such as oxidation, for which titanium is otherwise notorious. The optimal resistance for the above physical configuration is 90 to 150 angstroms of titanium. The actual choice of resistive film material does not alter the means and methods of this patent. Each material brings with it physical characteristics that can be compensated for using the general means of this patent.

(d) Nanoparticle Fabrication on a Resistive Substrate (U.S. patent application Ser. No. 10/854,420 which is incorporated by reference herein)

The palladium nanoparticles are fabricated on a resistive substrate by an electroplating method. The electroplating bath comprises 0.1 mM PdCl₂ and 0.1 M HCl dissolved in water. The process of electroplating the nanoparticles is necessary for successful operation of the sensor that nanoparticles have a certain distance between each other within a narrow distance window.

If inter-particle spacing is large, the sensor will be both slow and insensitive to low concentrations. Indeed, there will be a minimum threshold, for both temperature and concentration below which the sensor will not function. This is because the particles are spaced too far apart to touch each other, even at their times of greatest expansion and growth.

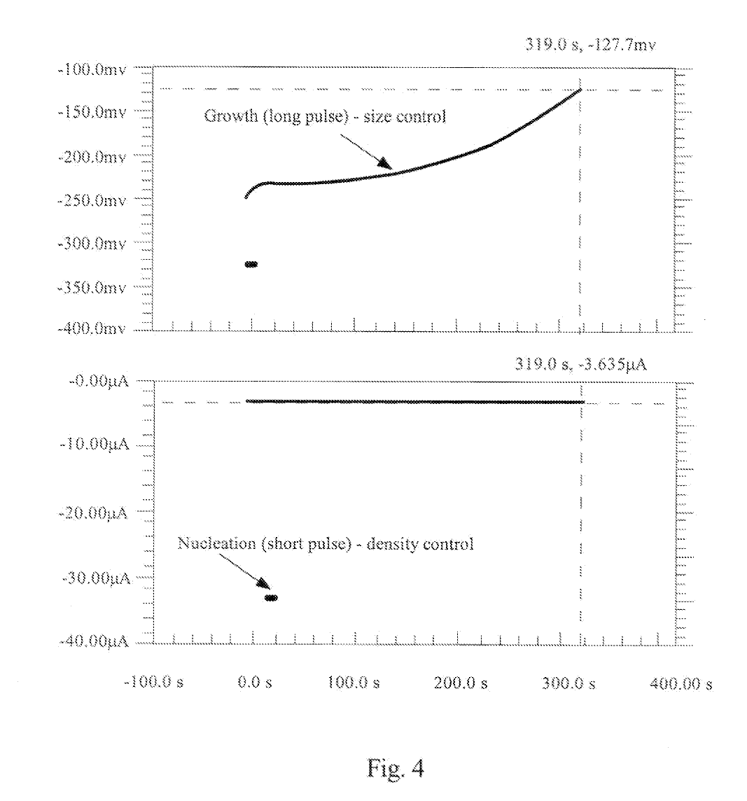

It is therefore necessary to control both the nano-particle size and the seeding density on the substrate. In this invention, palladium nanoparticles are grown by a two step plating process involving a short nucleation pulse (generally <10 sec) and a longer growth pulse (<10 minutes). The nucleation and growth parameters are controlled in the electrochemical fabrication process to produce functional sensors in different hydrogen concentration ranges. The density of the nanoparticles are generally controlled by the charge in the nucleation step (short pulse) and the size of the particles are controlled by the growth step (long pulse). A typical plating curve is shown in FIG. 4. A constant current process was employed for the electroplating process. The current paramerts substrate area dependent.

The speed of the sensor (referred to as response time) can be controlled by controlling the size of the nanoparticles.

Thus a problem to be solved is to find a range of particle size and density for a fast sensor. Disclosed herein is a range of particle size and density that achieves a response time of 10 seconds or lesser at high hydrogen concentrations.

Identification of a Range of Nanoparticle Size and Density for a Fast Responsive Hydrogen Sensor

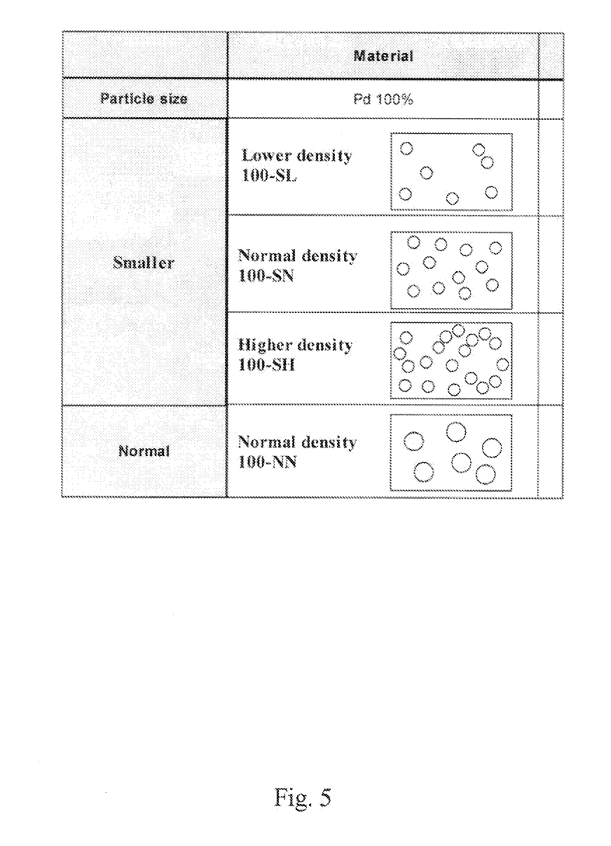

FIG. 5 shows a matrix where the particle size and density are varied during the electroplating process. Four Variations of the particle size and density were studied with the goal of identifying a sensor with the fastest response time. The experimental variations are given below:

### EXAMPLE 1

### Type—Smaller Size, Lower Density

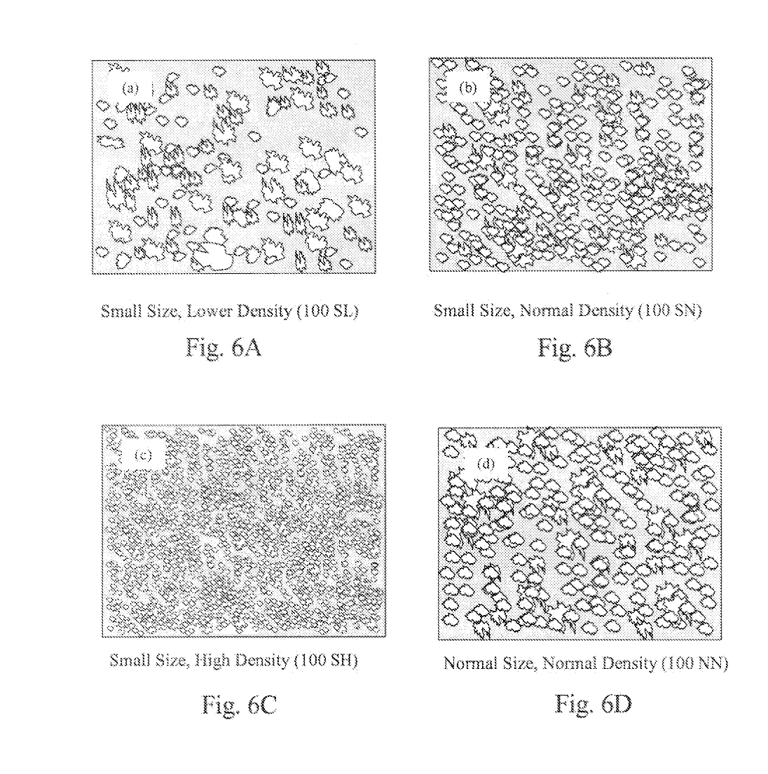

It can be seen that the (100-SL) sensors have a particle size of around 50 nm and an interparticle distance of around 150 nm. The SEM micrographs are shown in FIG. 6a. The nucleation time was decreased to provide lower particle density. The interparticle density was decreased by decreasing the nucleation current.

### EXAMPLE 2

### Type—Smaller Size, Normal Density

It can be seen that the (100-SN) sensors have a particle size of around 50 nm and an interparticle distance of around 30 nm. The SEM micrographs are shown in FIG. 6b. The nucleation current was maintained close to control parameter (the actual value of nucleation current is substrate area dependent in a constant current process) to provide a normal particle density.

### EXAMPLE 3

### Type—Smaller Size, Higher Density

It can be seen that the (100-SH) sensors have a particle size of around 20 nm and an interparticle distance of around 1-2 nm. The response time (t90) of the sensor was around 25 seconds for 400 ppm H₂. The SEM micrographs are shown in FIG. 6c. The particle size was decreased by decreasing the growth time and the interparticle density was increased by increasing the nucleation current.

### EXAMPLE 4

### Type—Normal Size, Normal Density

It can be seen that the (100-NN) sensors have a particle size of around 50 nm and an interparticle distance of around 30 nm. The response time (t90) of the sensor was around 35 seconds for 40000 ppm (4%) H₂. The SEM micrographs are shown in FIG. 6d. The nucleation and growth were maintained consistent with the control plating conditions to provide normal size and density.

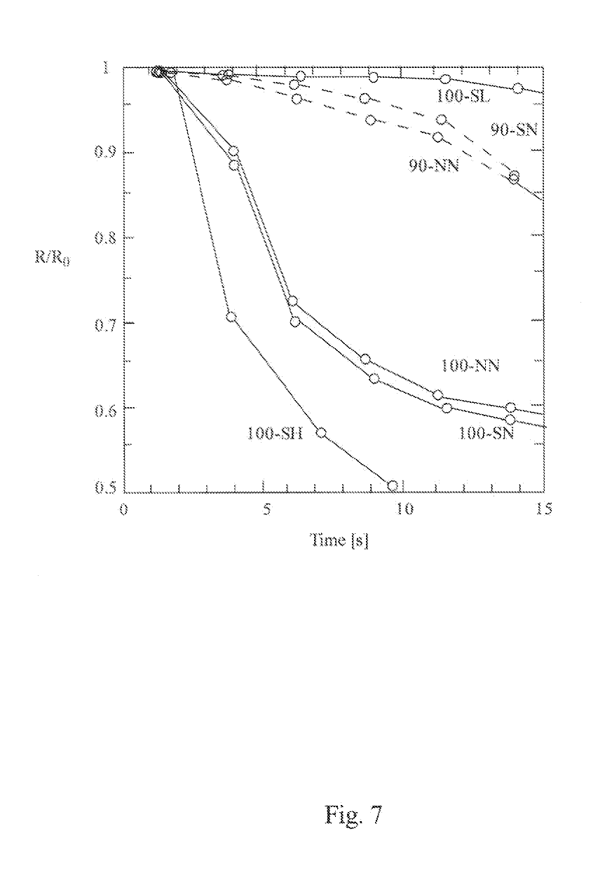

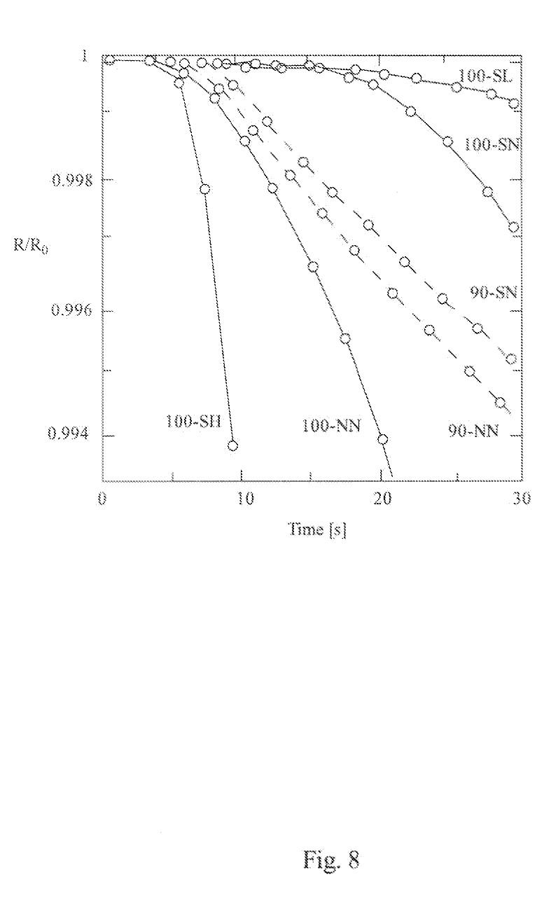

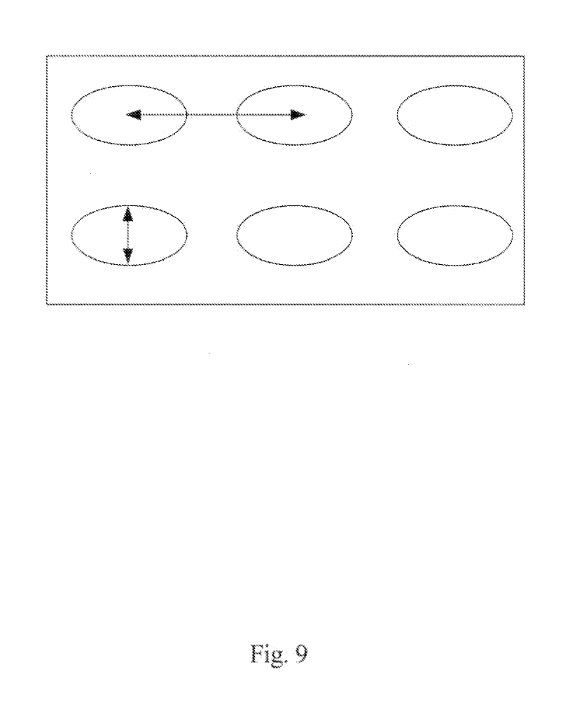

FIG. 7 shows the response of the four sensors to 40000 ppm H₂ and FIG. 8 shows the response of the four sensors to 400 ppm H₂. The small size, high density type (100-SH) has a response time of 10 seconds, while the normal size, normal density type (100-NN) has a response time of greater than 30 seconds. The particle interparticle distance (l) is calculated by the center to center distance between two adjacent particles. The ratio of particle diameter (d) to interparticle distance (l) is defined as the ratio between the diameter of any given particle divided by the center to center distance of between the adjacent particle as illustrated in the schematic in FIG. 9.

The ratio of particle diameter (d) to interparticle distance (l) of the 100-SH type is around 0.85 to 1.0 and that for the 100-NN type is around 0.6 to 0.85. Thus the particle diameter (d) to interparticle distance (l) of the nanoparticles determines the speed of sensor.

Thus, the particle size and densities were varied for pure Pd sensors to achieve a faster response time. Concluded is that a sensor with higher particle density and smaller size (100-SH) improves the sensor performance in terms of response time.

FIG. 11 shows the principle of a hydrogen sensor. The palladium or palladium composite particles is supported on base. Under hydrogen atmosphere, these particles are swelled to contact each other and the electrical properties between electrodes changes. For example, under constant current mode, the resistance between electrodes decreases when the sensor is exposed to hydrogen.

The hydrogen sensor may be made by a glass substrate cleaned and metal film deposited on it. After that, it is patterned and contact pads deposited. The detecting part of sensor is made through wafer dicing, electroplating and chip dicing. The whole unit of sensor may be about 1 cm×1 cm and detection part smaller than 0.5 cm×0.5 cm. The palladium or palladium-silver composite particles are supported on base. The particle size may be about 100 nm. The particle size and particle packing density may be varied as shown in Table 1. The composition of metal was 100% of palladium or the ratio of palladium and silver being 90:10. These particles were arranged as several belts of each width being 10 μm.

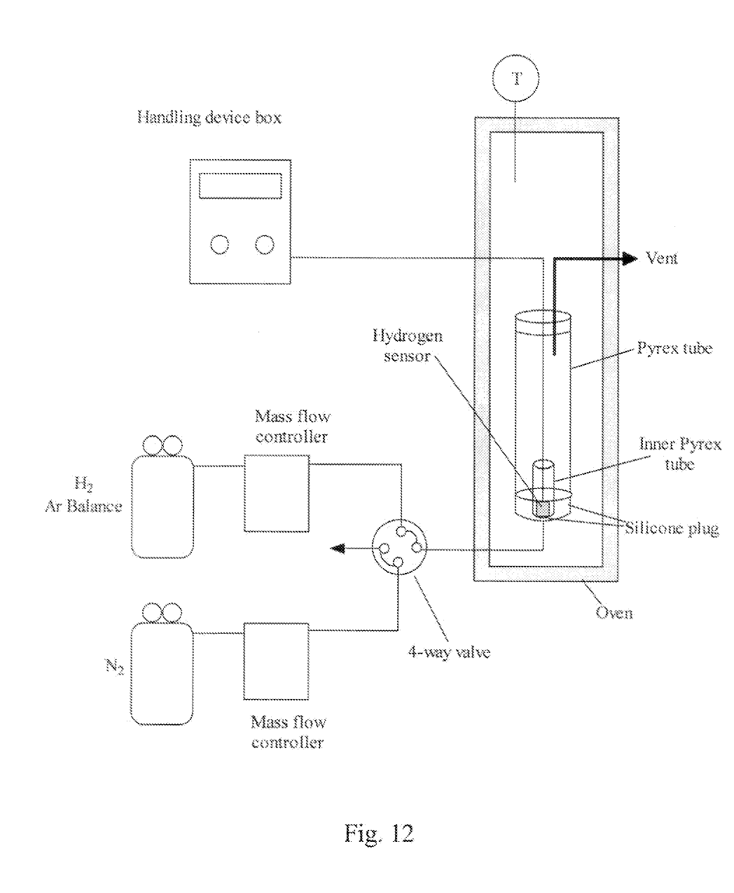

FIG. 12 shows an experimental apparatus. The hydrogen sensors are fixed in glass cell made from pyrex tube. The glass cell is placed in a column oven whose temperature is controlled at analysis temperature. At the entrance of glass cell, the smaller size of glass tube (3 cm long, 1.5 cm i.d.) is put to enhance the exchange of gases around the sensor. The test gases are 4% 4000 ppm and 400 ppm hydrogen diluted with argon. The nitrogen is also used as an inert gas. These gases are supplied with mass-flow controller. At first, 100 cc/min with a 4-way valve. After a certain period, the gas is changed to nitrogen. The electric signal from the sensor is monitored with a handling device box and the residence evaluated.

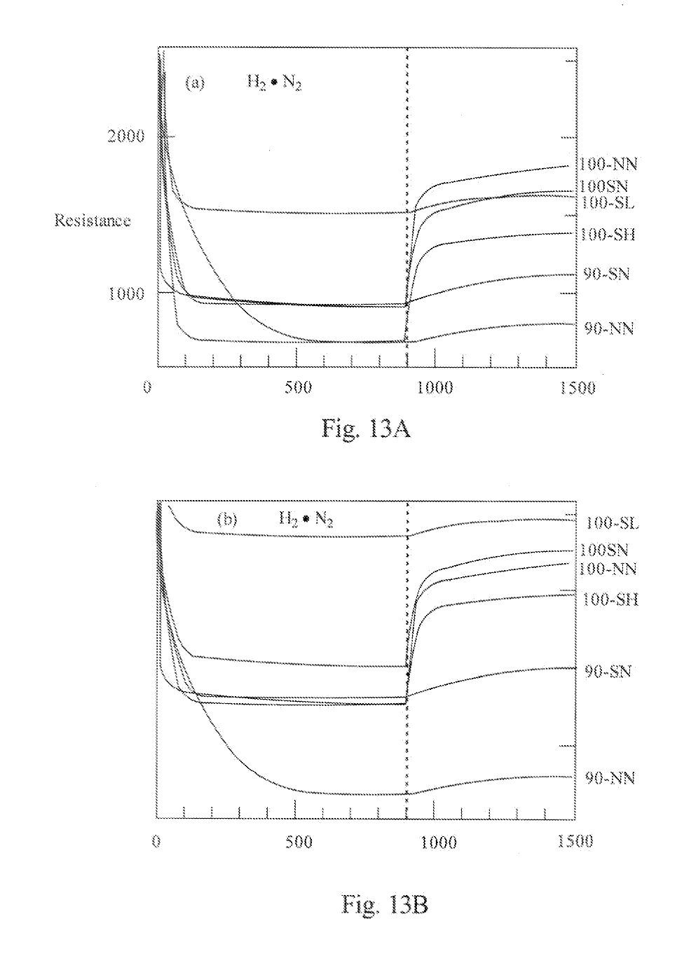

The performance of the hydrogen sensor was tested. FIG. 13 shows the change of resistance of hydrogen sensors at 333 K under 4% hydrogen. FIG. 13(a) shows absolute residence and FIG. 13(b) shows relative residence based on initial residence of sensor. After the gas switched from nitrogen to hydrogen, the resistance of the sensor rapidly decreased and then seems to be almost constant. The magnitude of change of relative residence under hydrogen was from 30 to 90% and was depended on the situation of particles. The pattern of palladium composite particles influenced the performance of the sensor. In particular, the resistance for 100-SH and 100-SN was almost half within 10 seconds of exposure time. After 900 seconds (15 minutes), the gas was switched from hydrogen to nitrogen. At that time, the resistance of sensor increased to initial value, but the speed for increase was lower than the speed for the decrease. These results indicate that the hydrogen readily permeated to palladium composite metal and desorption of hydrogen from palladium composite metal was slower than the permeation.

Material

| Particle Size | Pd 100% | Pd:Ag = 90:10 |
|---|---|---|
| Smaller | Lower density 100-SL | Normal density 90-SN |
| | Normal density 100-SN | |
| | Higher density 100-SH | |
| Normal | Normal density 100-NN | Normal density 90-NN |

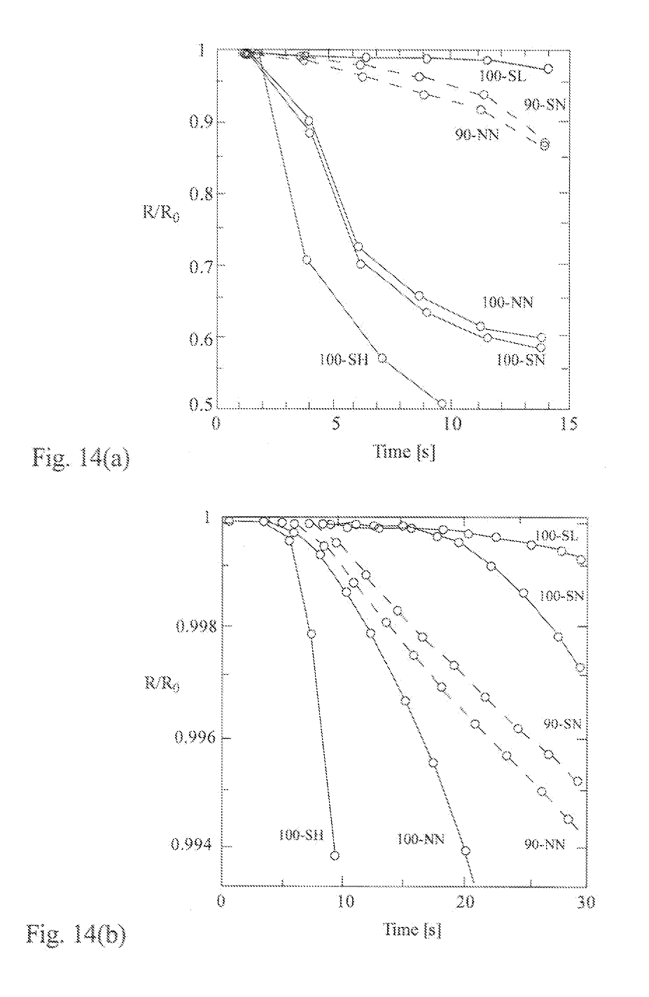

FIG. 14 shows the initial resistance of a sensor at 333 K. In the case of 4% hydrogen, the responsibility was in the order of 100-SH>100-SN, 100-NN>90-NN, 90-SN, 100-SL. In the case of 400 ppm hydrogen, that was in the order of 100-SH>100-NN>90-NN, 90-SN>100-SN>100-SL. The responsibility of 100-SH was the highest and that of 100-SL was the lowest regardless of hydrogen concentration, which means that the high particle packing density leaded to high responsibility. When the particle packing density is high, each particle was close to be easy to contact each other in swelling. The composition of metal affected the responsibility of sensors. In the case of 4% hydrogen, the responsibility of 100-SN and 100-NN was significantly higher than that 90-SN and 90-NN, respectively. In the case of 400 ppm hydrogen, the responsibility of 100-NN was higher than that of 90-NN and the responsibility of 90-SN was higher than 100-SN.

The relative order for 90-SN and 100-SN was not revealed. However, on the whole, the addition of silver would inhibit the embrittlement by hydrogen and the responsibility of sensor decreased. Next, the effect of particle size. In the case of 4% hydrogen, the responsibility was almost constant regardless of particle size (between 100-NN and 100-SN, 90-NN and 90-SN). In the case of 400 ppm hydrogen, the responsibility increased with the increasing particle size. In the particle size of this study, the large particle size seems to be desirable for high responsibility.

Above, the 100-SN type sensor shows the highest responsibility in any case. Next evaluated are the effect of temperature and hydrogen concentration of 100-SN type sensor in detail.

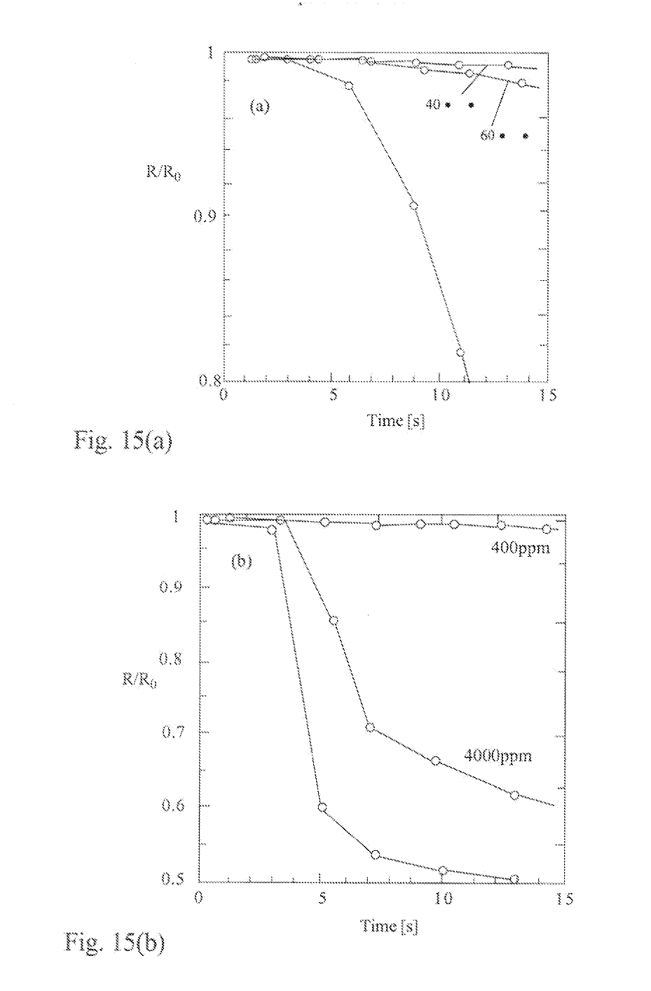

FIG. 15 shows the response of a 100-SN type sensor for temperature and hydrogen concentration. The responsibility considerably increased with increasing temperature (FIG. 15(a)).

In particular, the responsibility of 80° C. was significantly higher than that of 60° C. At 80° C., the relative difference of resistance was about 0.9 within 10 seconds. This high responsibility was because the increase of temperature probably made the diffusion rate of hydrogen atom in palladium composite metal higher and leaded to fast swelling of metal to give high responsibility of sensors.

FIG. 15(b) shows the response of a sensor for hydrogen concentration at 333 K. The magnitude of the change in resistance greatly increased with increasing hydrogen concentration. In general, diffusion rate of hydrogen in palladium metal is in proportion to the difference of partial pressure of hydrogen. The partial pressure of hydrogen is almost in proportion to hydrogen concentration. In the high hydrogen pressure region, the difference of partial pressure of hydrogen between inside of metal and metal surface is high. The effect of hydrogen concentration can be explained above principle.

Developed are several type hydrogen sensors by using palladium nano-particles, evaluated over a wide temperature range and hydrogen concentration. The sensor detected hydrogen by the change of resistance related to the swelling of palladium and the resistance of sensor decreased under hydrogen atmosphere. This hydrogen sensor detected hydrogen concentration over a range from 400 ppm to 4% regardless of the particle size and particle packing density. On the whole, the responsibility of the sensor made from 100% palladium was higher than that made from 90% palladium-10% silver composite. Further, the increase in particle packing density promoted the response of sensor. The increase in both temperature and hydrogen concentration significantly increased the responsibility of sensor, which is probably because the diffusion rate of hydrogen in palladium increases with temperature and the difference of partial pressure between inside and outside of particles.

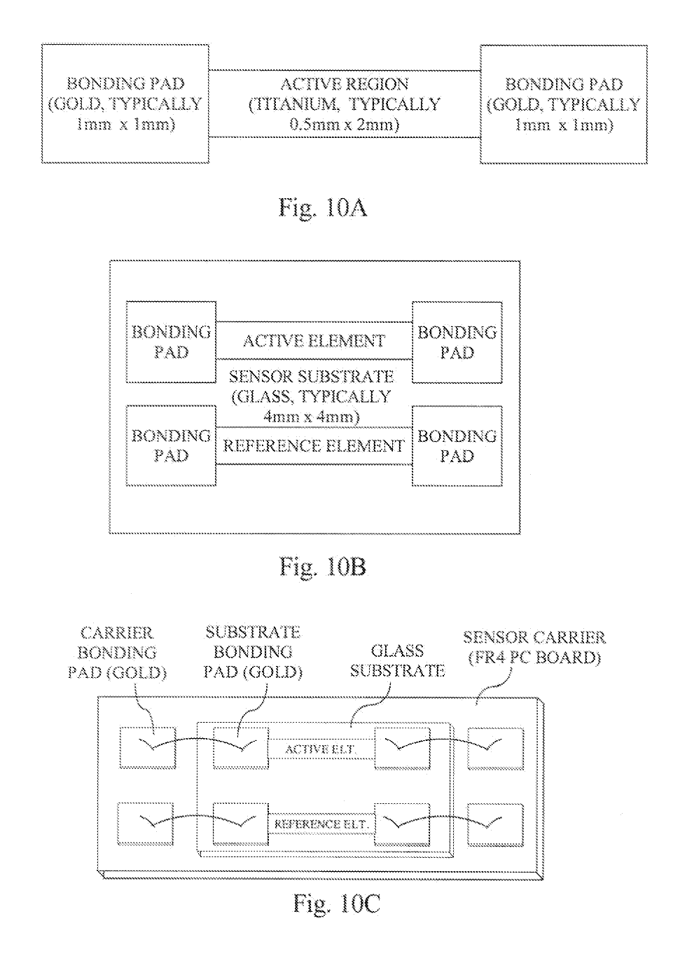

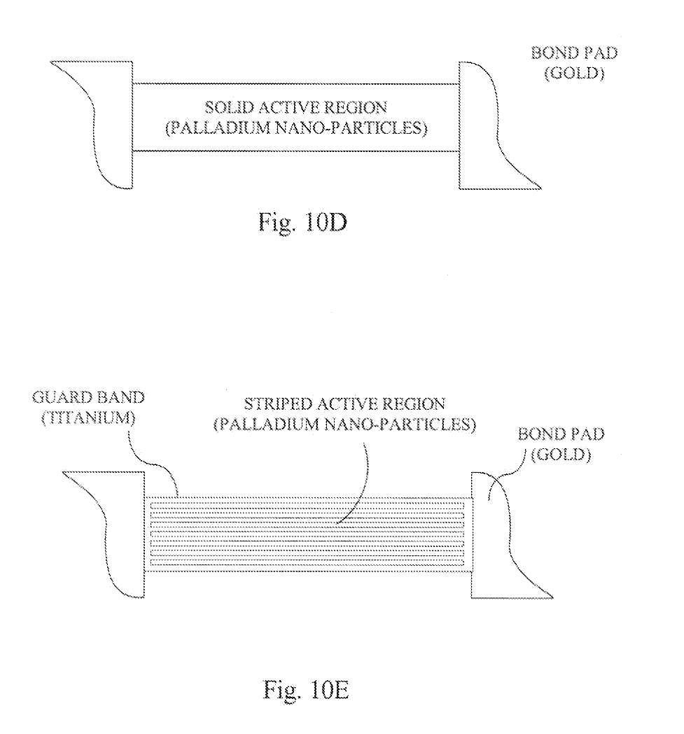

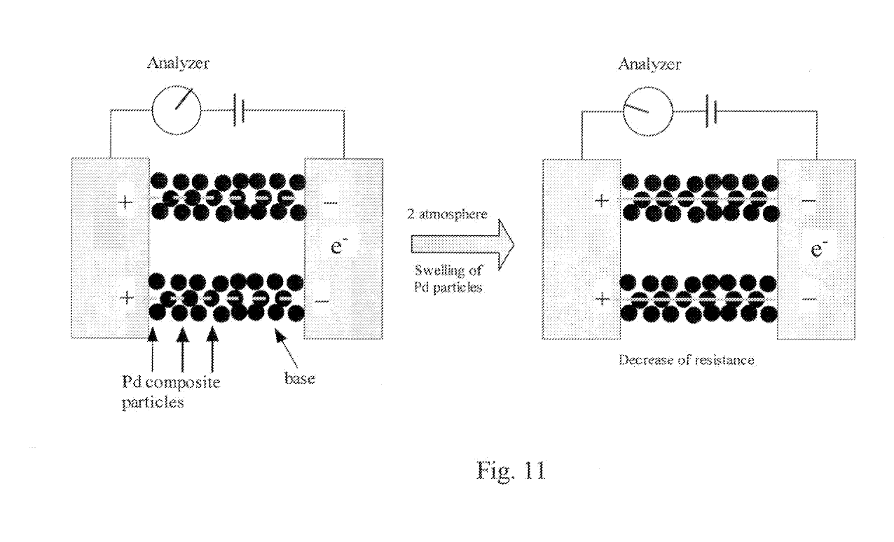

Referring to FIG. 10A, it has been found convenient to have a 0.5 mm×2 mm (Length/Diameter=4) active area on the sensors. Other sizes have been used, but this is a trade-off between resistance, active area and sensor stability. At each end of this area may be a 1 mm×1 mm gold bonding pad.

The substrate material may be titanium, although this may be replaced with less-reactive vanadium. One skilled in the art will appreciate that various other materials could be used, including organic materials, so long as they fit the resistivity and operational ranges, and material compatibility issues for the sensor as a whole.

Titanium is a quite reactive metal, and must be well understood to be useful in a sensor application such as this. Referring to FIG. 10B, to compensate for oxidation-based aging of the sensors, a reference resistive element may be added to the sensor. It may be identical to the active sensing element, but may be no palladium plating. Both oxidize at approximately the same rate, and the reference element is used to compensate for residual aging resistance changes.

To minimize oxidation-based aging in the field, the sensors may be pre-oxidized by subjecting them to an elevated temperature in an oxygen atmosphere. For example, the resistive Ti film may be 100 Angstroms thick when created. Oxidation may reduce that thickness to perhaps 80 Angstroms, for example, replacing 20 Angstroms by TiO₂, an insulator.

While the oxidation continues indefinitely, it gradually becomes a much slower process as the oxide thickens, because large O₂ molecules are required to penetrate far deeper than at the start of the process.

To control the aging, the Ti layer may therefore be thickened so that it can be corrected back by the thinning process of pre-oxidizing it. Therefore, thicker films of 150 Angstroms, for example, may be used instead of thinner 90 Angstroms, for example. The trade-off is that it provides a lower initial resistance. FIG. 10C illustrates the sensor pair mounted on a sensor carried PC board.

Referring to FIGS. 10B and 10C, a single sensor may comprise two elements, one active and one for reference. They may be identical in size and shape, except that the reference element is not plated. A 0.5 mm×2 mm resistive area may be used by way of example, but one skilled in the art will realize that other sizes and geometries can be used without altering the means of this invention.

Referring to FIG. 10D, the non-gold (non-pad) region of the active element of the sensor may be covered by a 20 μm mask border to preclude it from being plated. This prevents E-field effects from causing more aggressive plating near the edges of the element.

The reference element (FIG. 10B), may be identical in every way to the active element (FIG. 10B), except that it may not be placed with palladium. The photomask used to create the palladium plating windows may simply cover the entirety of the reference element during the plating step.

For the active element, two palladium mask types may be used, solid-fill (FIG. 10D) or striped (FIG. 10E). In the solid-fill version, except for the 20 μm borders, the entire active area is plated with palladium. In the “striped” version, various widths of palladium lines may be formed, all over a solid titanium resistive sheet. Nominal line-and-space widths may be 10 μm and 10 μm, respectively.

1. A sensor comprising nanoparticles that expand in a presence of hydrogen, the nanoparticles having a particle size less than 50 nanometers, the nanoparticles deposited on a substrate with a density where an average ratio of particle size to a distance between particle centers is in a range of 0.85 to 1.00.

2. The sensor as recited in claim 1, wherein the nanoparticles comprise palladium nanoparticles.

3. The sensor as recited in claim 1, wherein the nanoparticles are palladium nanoparticles.

4. The sensor as recited in claim 1, wherein the nanoparticles comprise palladium and silver.

5. The sensor as recited in claim 2, further comprising two electrodes positioned at ends of the substrate for sensing a current running through the substrate and nanoparticles.

6. The sensor as recited in claim 5, wherein the substrate is resistive.

7. The sensor as recited in claim 1, wherein the nanoparticles have a particle size in a range of 20-30 nanometers.

8. The sensor as recited in claim 1, wherein the sensor is operated in a temperature range of 0 to 100C.

9. The sensor as recited in claim 1, wherein the sensor is operated in a temperature range of 60C. to 90C.
